(19) United States
(12) Patent Application Publication
(10) Pub. No.: US 2012/0232390 A1
(43) Pub. Date: Sep. 13, 2012

(54) DIAGNOSTIC APPARATUS AND METHOD

(75) Inventor: Moon-ho Park, Seoul (KR)

(73) Assignee: SAMSUNG ELECTRONICS CO., LTD., Suwon-si (KR)

(21) Appl. No.: 13/296,550

(22) Filed: Nov. 15, 2011

(30) Foreign Application Priority Data
Mar. 8, 2011 (KR) 10-2011-0020619

(51) Int. Cl. A61B 8/14

(52) U.S. Cl. 600/443

(57) ABSTRACT

A diagnostic apparatus and method is provided. A diagnostic apparatus includes a region of interest (ROI) detection unit configured to detect at least one ROI in a diagnostic image formed according to an echo signal returned from a subject, an emphatic image generation unit configured to generate an emphatic image, and a display unit configured to display the generated emphatic image.

### CROSS-REFERENCE TO RELATED APPLICATIONS

This application claims the benefit of Korean Patent Application No. 10-2011-0020619, filed on Mar. 8, 2011, in the Korean Intellectual Property Office, the entire disclosure of which is incorporated herein by reference for all purposes.

### BACKGROUND

1. Field

The following description relates to diagnostic apparatuses and methods.

2. Description of the Related Art

In ultrasonic medical imaging, a medical diagnostic image showing the size, structure, or pathologic damage of a human organ may be generated in real time using an ultrasonic signal. Compared to computed tomography (CT) or magnetic resonance imaging (MRI), ultrasonic diagnosis is harmless to the human body because ionizing radiation, which is harmful to the human body and may cause cancer or gene disruption, is not used. Further, because it is noninvasive in imaging human organs, relatively inexpensive, and can be performed by using easily-movable equipment, ultrasonic diagnosis may be broadly used.

### SUMMARY

In one general aspect, there is provided a diagnostic apparatus, including a region of interest (ROI) detection unit configured to detect at least one ROI in a diagnostic image formed according to an echo signal returned from a subject, an emphatic image generation unit configured to automatically generate an emphatic image in which a resolution of the detected ROI is improved, and a display unit configured to display the generated emphatic image.

The general aspect of the diagnostic apparatus may further include that the ROI detection unit configured to detect a plurality of ROIs, and the emphatic image generation unit is further configured to generate the emphatic image in which the resolution of each of the detected ROIs is improved according to different ratios corresponding to a level of a feature of representing whether a tissue included in each of the ROIs has a lesion.

The general aspect of the diagnostic apparatus may further include that the emphatic image generation unit is further configured to generate the emphatic image in which the resolution of the detected ROI is increased to a higher ratio if a probability is high that a tissue included in the detected ROIs has a lesion.

The general aspect of the diagnostic apparatus may further include a lesion determination unit configured to determine whether a first tissue included in a first ROI has a lesion in the diagnostic image, the emphatic image, or a combination thereof with respect to each of the detected ROI, and determine whether the first tissue has a lesion by using a determined result.

The general aspect of the diagnostic apparatus may further include that the lesion determination unit includes a first determination unit configured to determine whether the first tissue has a lesion by using a first feature value indicating a level of a feature representing whether the first tissue has a lesion in the diagnostic image, a second determination unit configured to determine whether the first tissue has a lesion by using a second feature value indicating a level of a feature representing whether the first tissue has a lesion in at least one of the diagnostic image and the emphatic image, and a third determination unit configured to determine whether the first tissue has a lesion by mixing the first and second feature values according to a determination ratio if a result of the first determination unit is different from a result of the second determination unit.

The general aspect of the diagnostic apparatus may further include that the second determination unit is further configured to determine whether the first tissue has a lesion by using two or more emphatic images.

The general aspect of the diagnostic apparatus may further include that the second determination unit includes a plurality of resolution-relevant classifiers configured to classify the first tissue as having a lesion or having no lesion in correspondence with each of a plurality of available resolutions of the ROI included in the emphatic image.

The general aspect of the diagnostic apparatus may further include that the second determination unit includes a resolution-irrelevant classifier configured to extract features representing whether the first tissue has a lesion commonly from the diagnostic image and the emphatic image, and classify the first tissue as having a lesion or having no lesion by using the extracted features.

The general aspect of the diagnostic apparatus may further include a database configured to store information regarding features for detecting the ROI, and a database management unit configured to add to the database information representing that a feature of the tissue does not correspond to the ROI if the lesion determination unit determines that the tissue has no lesion.

The general aspect of the diagnostic apparatus may further include that the lesion determination unit automatically determines whether the tissue included in the detected ROI has a lesion.

The general aspect of the diagnostic apparatus may further include that the display unit configured to display a result of the determination of the lesion determination unit together with the emphatic image.

In another aspect, there is provided a diagnostic method, including detecting at least one region of interest (ROI) in a diagnostic image formed according to an echo signal returned from a subject, automatically generating an emphatic image in which a resolution of the detected ROI is improved, and displaying the generated emphatic image.

The general aspect of the diagnostic method may further include that the detecting of the ROI includes detecting a plurality of ROIs, and the automatic generating of the emphatic image includes automatically generating the emphatic image in which the resolutions of each of the detected ROIs is improved according to different ratios corresponding to a level of a feature representing whether a tissue included in each of the ROIs has a lesion.

The general aspect of the diagnostic method may further include that the automatic generating of the emphatic image includes automatically generating the emphatic image in which the resolution of the detected ROI is increased to a higher ratio if a probability is high that a tissue included in the detected ROI has a lesion.

The general aspect of the diagnostic method may further include determining whether a first tissue included in a first ROI has a lesion in the diagnostic image, the emphatic image, or a combination thereof with respect to each of the detected ROI, and determining whether the first tissue has a lesion by using a determined result.

The general aspect of the diagnostic method may further include determining whether the first tissue has a lesion by using a first feature value indicating a level of a feature representing whether the first tissue has a lesion in the diagnostic image, determining whether the first tissue has a lesion by using a second feature value indicating a level of a feature representing whether the first tissue has a lesion in the diagnostic image, and the emphatic image, or a combination thereof, and determining whether the first tissue has a lesion by mixing the first and second feature values according to a determination ratio, if a result obtained by using the first feature value is different from a result obtained by using the second feature value.

The general aspect of the diagnostic method may further include that the determining of whether the first tissue has a lesion by using the second feature value includes determining whether the first tissue has a lesion by using two or more emphatic images.

The general aspect of the diagnostic method may further include that the determining of whether the first tissue is a lesion by using the second feature value includes determining whether the first tissue is a lesion by using a plurality of resolution-relevant classifiers configured to classify the first tissue as having a lesion or having no lesion in correspondence with each of a plurality of available resolutions of the ROI included in the emphatic image.

The general aspect of the diagnostic method may further include that the determining of whether the first tissue has a lesion by using the second feature value includes determining whether the first tissue has a lesion by using a resolution-irrelevant classifier configured to extract features representing whether the first tissue has a lesion commonly from the diagnostic image and the emphatic image, and classify the first tissue as having a lesion or having no lesion by using the extracted features.

In still another aspect, there is provided a computer readable recording medium having recorded thereon a computer program for executing the diagnostic method.

Other features and aspects may be apparent from the following detailed description, the drawings, and the claims.

### BRIEF DESCRIPTION OF THE DRAWINGS

FIG. 1 is a block diagram illustrating an example of a diagnostic apparatus according to a general aspect.

FIG. 2 illustrates an example of an emphatic image displayed on a display unit illustrated in FIG. 1.

FIG. 3 is a detailed block diagram illustrating an example of the diagnostic apparatus illustrated in FIG. 1.

FIG. 4 is a block diagram illustrating an example of a second determination unit illustrated in FIG. 3.

FIG. 5 is a block diagram illustrating another example of the second determination unit illustrated in FIG. 3.

FIG. 6 is a flowchart illustrating an example of a diagnostic method according to a general aspect.

FIG. 7 is a flowchart illustrating an example of a diagnostic method according to another general aspect.

Throughout the drawings and the detailed description, unless otherwise described, the same drawing reference numerals will be understood to refer to the same elements, features, and structures. The relative size and depiction of these elements may be exaggerated for clarity, illustration, and convenience.

### DETAILED DESCRIPTION

The following detailed description is provided to assist the reader in gaining a comprehensive understanding of the methods, apparatuses, and/or systems described herein. Accordingly, various changes, modifications, and equivalents of the systems, apparatuses, and/or methods described herein will be suggested to those of ordinary skill in the art. Also, descriptions of well-known functions and constructions may be omitted for increased clarity and conciseness.

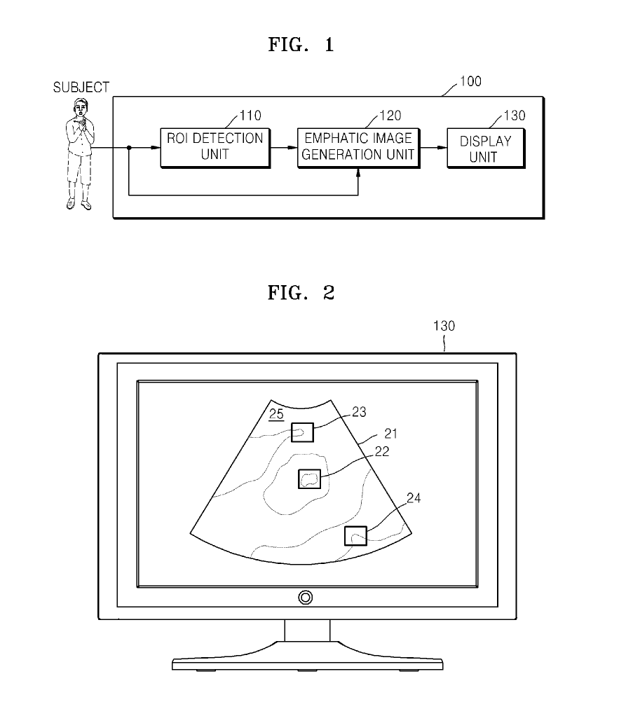

FIG. 1 is a block diagram illustrating an example of a diagnostic apparatus 100 according to a general aspect. Referring to FIG. 1, the diagnostic apparatus 100 includes a region of interest (ROI) detection unit 110, an emphatic image generation unit 120, and a display unit 130.

Elements related to the current example are illustrated in FIG. 1. Accordingly, the diagnostic apparatus 100 may further include other general-use components in addition to the illustrated elements.

In addition, the ROI detection unit 110 and the emphatic image generation unit 120 of the diagnostic apparatus 100 may include one processor or a plurality of processors. Each processor may be realized as an array of a plurality of logic gates, or a combination of a general-use microprocessor and a memory for storing a program executable in the microprocessor. Furthermore, it may be understood by one of ordinary skill in the art that the processor may be realized as another type of hardware.

The diagnostic apparatus 100 is an apparatus enabling the diagnosis of a subject. The subject may be, but is not limited to, a human body, or a liver, breast, or abdomen of a person.

The ROI detection unit 110 detects at least one ROI in a diagnostic image formed according to an echo signal returned from the subject. Further, the echo signal returned from the subject may be, but is not limited to, an ultrasonic signal.

The ROI represents a region that a user of the diagnostic apparatus 100 is interested in and desires to observe. Further, the user of the diagnostic apparatus 100 may be, but is not limited to, a medical professional such as a doctor or a nurse.

For example, the ROI may represent a lesion candidate region including a tissue suspected of having a lesion. For convenience of explanation, it will be described hereinafter that the ROI includes one tissue. However, the current embodiment is not limited thereto and the ROI may include a plurality of tissues.

In addition, it may be known to one of ordinary skill in the art that the lesion may include a malignant tumor, a malignant mass, or microcalcification.

As such, the ROI may be a region including a tissue that possibly has a lesion, i.e., a region including a tissue that is possibly not benign.

The ROI detection unit 110 may detect the ROI in the diagnostic image formed according to the echo signal returned from the subject by referring to a database (not shown) for storing information regarding the ROI.

The ROI detection unit 110 may detect the ROI in consideration of pixel values in the diagnostic image by using a binarization method. However, the current example is not limited thereto. A ROI detection method of the ROI detection unit 110 may be known to one of ordinary skill in the art, and thus a detailed description thereof is not provided here.

In addition, the ROI detection unit 110 may calculate a feature value indicating a level of a feature representing whether a tissue included in each ROI has a lesion. Also, the feature value may indicate a probability of a feature of a tissue included in each ROI representing a lesion. The calculating of the feature value will be described in detail later with reference to the emphatic image generation unit 120.

If the ROI detection unit 110 detects the ROI in the diagnostic image, the emphatic image generation unit 120 automatically generates an emphatic image in which the resolution of the ROI included in the diagnostic image is improved.

For example, the emphatic image generation unit 120 generates the emphatic image in which the resolution of the ROI included in the diagnostic image is higher than the resolution of a non-ROI (hereinafter referred to as a ‘normal region’).

Further, it may be known to one of ordinary skill in the art that the diagnostic apparatus 100 may additionally receive the echo signal from the subject one or more times in order to improve the resolution of the ROI. In addition, the additionally received echo signal may be a signal transmitted and returned in focus on the ROI of the subject, and thus may include information regarding the ROI.

Accordingly, the emphatic image generation unit 120 may additionally obtain an echo signal including the information regarding the ROI, and may use the obtained echo signal to generate the resolution-improved emphatic image.

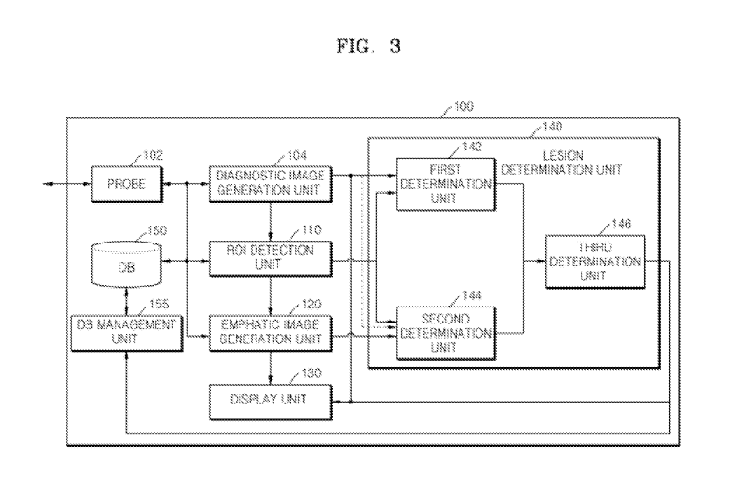

The emphatic image in which the resolution of the ROI is improved will be described in detail later with reference to FIG. 2.

Also, if the ROI detection unit 110 detects the ROI, the emphatic image generation unit 120 automatically generates the emphatic image. In the current example, the automatic generating of the emphatic image refers to automatically generating the emphatic image without a feedback, an involvement, or an additional manipulation of the user of the diagnostic apparatus 100.

In addition, the ROI detection unit 110 may detect a plurality of ROIs, and thus, the emphatic image generation unit 120 may generate an emphatic image in which the resolutions of the ROIs are improved to different ratios according to a level of a feature representing whether a tissue included in each of the ROIs has a lesion.

Further, the feature representing whether a tissue included in the ROI has a lesion may include a size, a shape, a margin, and a calcification level of the tissue.

For example, the shape of a tissue may be classified into a round, oval, lobulated, or irregular shape, and a probability that a tissue has a lesion is high if the shape of the tissue changes from a round shape to an irregular shape.

As another example, a probability that a tissue has a lesion is high if the margin of the tissue is unclear, microlobulated, stellate, or spiculated.

As still another example, when a tissue is calcified, if the tissue has a size equal to or less than about 0.5 mm, has a distribution equal to or greater than 5 pcs/cm³ in a group, has various sizes or pleomorphic shapes, has an irregular shape, or visually has a linear or branch-shaped distribution, a probability that the tissue has a lesion is high.

As such, the emphatic image generation unit 120 may generate an emphatic image in which each of the resolution of a plurality of ROIs is improved to a different ratio according to a level of a feature representing whether a tissue included in each of the ROIs has a lesion.

As mentioned above, a probability is high that a tissue included in the ROI has a lesion if the shape of the tissue changes from a round shape to an irregular shape. Accordingly, the emphatic image generation unit 120 increases the resolution of the ROI to a higher ratio if the tissue included in the ROI has an irregular shape.

Although only an example of the shape of a tissue is described above, the current example is not limited thereto. For example, the emphatic image generation unit 120 may increase the resolution of the ROI to a higher ratio if, in consideration of a plurality of features representing whether the tissue included in the ROI has a lesion, a high probability exists that a tissue included in the ROI has a lesion.

For example, a feature value indicating a level of a feature representing whether a tissue has a lesion may be set as a value equal to or greater than 0 and equal to or less than 5. That is, a probability that a tissue included in the ROI has a lesion may be represented as a value equal to or greater than 0 and equal to or less than 5 in consideration of a plurality of features representing whether the tissue has a lesion. Further, a feature value 0 represents that the probability that the tissue has a lesion is relatively low, and a feature value 5 represents that the probability that the tissue has a lesion is relatively high.

For example, when one or more ROIs detected by the ROI detection unit 110 includes a first ROI and a second ROI, if a feature value regarding a shape is 3 and a feature value regarding a margin is 4 with respect to a first tissue included in the first ROI, a feature value indicating a level of a feature representing whether the first tissue has a lesion may be an average value of the feature values regarding the shape and the margin, i.e., 3.5.

Also, if a feature value regarding a shape is 5 and a feature value regarding a margin is 4 with respect to a second tissue included in the second ROI, a feature value indicating a of a feature representing whether the second tissue has a lesion may be an average value of the feature values regarding the shape and the margin, i.e., 4.5.

Further, the emphatic image generation unit 120 may generate the emphatic image for the first and second ROIs in resolutions corresponding to the respective feature values of the first and second tissues of the first and second ROIs. For example, an emphatic image generated of the first ROI has a higher resolution than the resolution of an emphatic image generated of the normal region. Further, since the feature value of the second ROI is greater than the feature value of the first ROI, an emphatic image generated of the second ROI has a resolution that is higher than the resolution of the emphatic image generated of the first ROI.

A feature value indicating a level of a feature of a tissue included in each ROI may be determined by the ROI detection unit 110. However, the current example is not limited thereto, and the feature value may be determined by the emphatic image generation unit 120.

In addition, if there is a high probability that a tissue included in each of the ROIs has a lesion, the emphatic image generation unit 120 generates the emphatic image in which the resolution of the ROI is increased to a high ratio.

For example, if a probability is less than 50% that a tissue included in the ROI has a lesion, the emphatic image generation unit 120 may generate the emphatic image in which the resolution of the ROI is increased to a level that is two times the resolution of the normal region. In addition, if a probability is equal to or greater than 80% that a tissue included in the ROI has a lesion, the emphatic image generation unit 120 may generate the emphatic image in which the resolution of the ROI is increased to a level that is eight times the resolution of the normal region.

As such, the resolution of the ROI may be increased to a high ratio if a probability is high that a tissue included in the ROI has a lesion, and thus, may result in an improved accuracy of diagnosis.

In addition, the emphatic image generation unit 120 may generate a plurality of emphatic images in which the resolution of the ROI is increased to different ratios. The emphatic images may be automatically generated according to a setup option.

Further, the generated emphatic images may be sequentially converted and displayed on the display unit 130. In addition, emphatic images may be converted automatically or according to a manipulation of the user.

For example, the emphatic image generation unit 120 may generate a first emphatic image in which the resolution of the ROI is increased four times higher than the resolution of the normal region, and a second emphatic image in which the resolution of the ROI is increased eight times higher than the resolution of the normal region.

As another example, the emphatic image generation unit 120 may generate a first emphatic image in which the resolution of the first ROI is increased twice higher than the resolution of the normal region and the resolution of the second ROI is increased three times higher than the resolution of the normal region, and a second emphatic image in which the resolution of the first ROI is increased four times higher than the resolution of the normal region and the resolution of the second ROI is increased six times higher than the resolution of the normal region

As such, the emphatic image generation unit 120 may automatically generate various emphatic images in consideration of convenience of the user.

As described above, the emphatic image generation unit 120 may generate emphatic images having various resolutions with respect to ROIs in consideration of levels of interest of the user, and, thus, may serve to improve convenience and accuracy of diagnosis. In addition, since the emphatic image generation unit 120 automatically generates the emphatic images, the emphatic image generation unit 120 may serve to reduce the time and effort required for manually controlling the diagnostic apparatus 100.

The display unit 130 displays the emphatic image generated by the emphatic image generation unit 120. The display unit 130 includes an output device included in the diagnostic apparatus 100, e.g., a display panel, a touch screen, a liquid crystal display (LCD) screen, or a monitor, and software for driving the output device.

Accordingly, the diagnostic apparatus 100 may generate and display an emphatic image in which the resolution of an ROI required to be attentively observed in a diagnostic process is automatically improved, and, thus, may serve to improve the convenience and accuracy of diagnosis of a user of the diagnostic apparatus 100.

In addition, the diagnostic apparatus 100 may diagnose a subject by using, but is not limited to, a computer aided diagnosis (CAD) method or a multi-level CAD method. The CAD method automatically detects and diagnoses a lesion by using a computer to analyze a medical image and patient data. The CAD method may serve to improve the accuracy in a determination of a lesion.

FIG. 2 illustrates an example of an emphatic image 21 displayed on the display unit 130 illustrated in FIG. 1. Referring to FIGS. 1 and 2, the display unit 130 displays the emphatic image 21.

For example, if the ROI detection unit 110 detects a first ROI 22, a second ROI 23, and a third ROI 24 in a diagnostic image, the emphatic image generation unit 120 automatically generates an emphatic image in which the resolutions of the first through third ROIs 22 through 24 are improved.

If the resolution of the diagnostic image is ‘a’, the resolution of a normal region 25 in the emphatic image is also ‘a’. Further, the resolutions of the first through third ROIs 22 through 24 may be ‘b’, wherein ‘a<b’.

Also, for example, if a level of a representing whether a first tissue included in the first ROI 22 has a lesion has the smallest value and a level of a feature representing whether a third tissue included in the third ROI 24 has a lesion has the largest value, when the resolution of the diagnostic image is ‘a’, the resolution of the normal region 25 in the emphatic image is also ‘a’. Further, the resolutions of the first through third ROIs 22 through 24 may respectively be ‘b’, ‘c’, and ‘d’, wherein ‘a≦b≦c<d’.

Accordingly, the emphatic image generation unit 120 may generate the emphatic image in which the resolutions of the first through third ROIs 22 through 24 are improved, and the generated emphatic image may be displayed on the display unit 130. In addition, as illustrated in FIG. 2, the size of the ROI is not changed even when the resolution of the ROI is improved. Thus, the user may easily identify the ROI and the normal region and may diagnose a subject accurately.

FIG. 3 is a detailed block diagram illustrating an example of the diagnostic apparatus 100 illustrated in FIG. 1. Referring to FIG. 3, the diagnostic apparatus 100 includes a probe 102, a diagnostic image generation unit 104, the ROI detection unit 110, the emphatic image generation unit 120, the display unit 130, a lesion determination unit 140, a database 150, and a database management unit 155. The lesion determination unit 140 includes a first determination unit 142, a second determination unit 144, and a third determination unit 146.

Elements related to the current example are illustrated in FIG. 3. Accordingly, it may be understood by one of ordinary skill in the art that the diagnostic apparatus 100 may further include other general-use components in addition to the illustrated elements.

The diagnostic apparatus 100 illustrated in FIG. 3 is an example of the diagnostic apparatus 100 illustrated in FIG. 1. As such, the diagnostic apparatus 100 is not limited to the elements illustrated in FIG. 3. In addition, the above descriptions provided in relation to FIG. 1 are also applicable to FIG. 3 and thus repeated descriptions are not provided here.

The probe 102 transmits and receives a signal to and from a subject. Further, the transmitted and received signal may be, but is not limited to, an ultrasonic signal. The probe 102 converts an electrical signal into an ultrasonic signal by using a transducer. The probe 102 transmits the ultrasonic signal to the subject and reconverts the ultrasonic signal returned from the subject into the electrical signal.

In addition, it may be known to one of ordinary skill in the art that the probe 102 may include a beamformer for controlling a delay time of the signal transmitted to and received from the subject. As such, the probe 102 may convert the ultrasonic signal returned from the subject into the electrical signal, and may form a reception beam by using the converted electrical signal, the reception beam being used to generate a diagnostic image.

An echo signal returned from the subject may include the ultrasonic signal returned from the subject, the electrical signal converted from the returned ultrasonic signal, and the reception beam used to generate the diagnostic image.

Also, in order to allow the emphatic image generation unit 120 to generate an emphatic image in which the resolution of an ROI is improved, the probe 102 may additionally receive the echo signal from the subject one or more times. For this, the probe 102 may transmit a signal focused on the ROI detected by the ROI detection unit 110. Further, the probe 102 may transmit and receive the signal focused on the ROI by adjusting parameters such as a gain, a dynamic range, sensitivity time control (STC)/time gain compensation (TGC), the number and positions of focuses, and a depth of focus of the transmitted signal. The probe 102 may automatically adjust the parameters if the ROI detection unit 110 detects the ROI.

A signal transmitting and receiving method of the probe 102 may be known to one of ordinary skill in the art, and thus, a detailed description thereof is not provided here.

The diagnostic image generation unit 104 generates a diagnostic image by using the echo signal returned from the subject. The diagnostic image generation unit 104 may include a digital signal processor (DSP) (not shown) and a digital scan converter (DSC) (not shown). The DSP forms image data representing a ‘b’, ‘c’, or ‘d’ mode by processing a signal output from the probe 102, and the DSC generates a scan-converted diagnostic image to display the image data formed by the DSP.

The ROI detection unit 110 detects one or more ROIs in the diagnostic image generated by the diagnostic image generation unit 104. In addition, the ROI detection unit 110 may calculate a feature value indicating a level of a feature representing whether a tissue included in each ROI has a lesion.

If the ROI detection unit 110 detects the ROI, the emphatic image generation unit 120 automatically generates an emphatic image in which the resolution of the ROI included in the diagnostic image generated by the diagnostic image generation unit 104 is improved.

Further, the emphatic image generation unit 120 may additionally receive the echo signal from the probe 102 to generate the emphatic image. That is, the emphatic image generation unit 120 may automatically and additionally receive the echo signal including information regarding the ROI detected by the ROI detection unit 110, and may automatically generate the emphatic image with reference to the additionally received echo signal.

Like the diagnostic image generation unit 104, the emphatic image generation unit 120 may include a DSP (not shown) and a DSC (not shown).

The display unit 130 displays the diagnostic image generated by the diagnostic image generation unit 104, the emphatic image generated by the emphatic image generation unit 120, a determination result of the lesion determination unit 140, or any combination thereof.

For example, the display unit 130 may display the diagnostic image, the emphatic image, or the emphatic image and information showing whether a lesion is included in the emphatic image.

The lesion determination unit 140 determines whether a first tissue included in a first ROI in the diagnostic image, the emphatic image, or a combination thereof has a lesion with respect to each ROI detected by the ROI detection unit 110, and determines whether the first tissue has a lesion by using a determination result. In addition, the lesion determination unit 140 may automatically determine whether the tissue included in the ROI has a lesion if the ROI detection unit 110 detects the ROI.

The ROI detected by the ROI detection unit 110 includes a first ROI, and the first ROI includes a first tissue. Hereinafter, for convenience of explanation, the first ROI and the first tissue included in the first ROI will be representatively described. However, the following descriptions may be applied to each ROI detected by the ROI detection unit 110 and the tissue included in the ROI.

The lesion determination unit 140 includes the first through third determination units 142, 144, and 146. In addition, each of the first and second determination units 142 and 144 may classify the first tissue as having a lesion or having no lesion by using a classifier using a CAD method. However, the current example is not limited thereto.

The first determination unit 142 determines whether the first tissue has a lesion by using a first feature value indicating a level of a feature representing whether the first tissue has a lesion in the diagnostic image. Further, the first feature value may be calculated by the ROI detection unit 110 or the diagnostic image generation unit 104, or may be determined by the first determination unit 142.

As such, the first determination unit 142 compares the first feature value to a threshold value for determining a lesion and determines that the first tissue has a lesion if the first feature value is greater than the threshold value. For example, a classifier included in the first determination unit 142 may classify the first tissue as having a lesion.

On the other hand, the first determination unit 142 determines that the first tissue has not a lesion if the first feature value is equal to or less than the threshold value. For example, the classifier included in the first determination unit 142 may classify the first tissue as having no lesion.

Further, the classifier included in the first determination unit 142 may be a classifier using a CAD method. As such, the classifier included in the first determination unit 142 may adjust the threshold value by using learned data. For example, the classifier may adaptively adjust the threshold value according to learned data by using a statistical pattern recognition method such as multi-layer perception (MLP). In addition, although the classifier uses one threshold value in the above description for convenience of explanation, the classifier is not limited thereto and may use a two-dimensional line or a three-dimensional plane as a reference of classification.

The classifier using learned data in the CAD method may be known to one of ordinary skill in the art that, and thus, a detailed description thereof is not provided here.

The second determination unit 144 determines whether the first tissue has a lesion by using a second feature value indicating a level of a feature representing whether the first tissue has a lesion in the diagnostic image, the emphatic image, or a combination thereof. Further, the second feature value may be determined by the second determination unit 144.

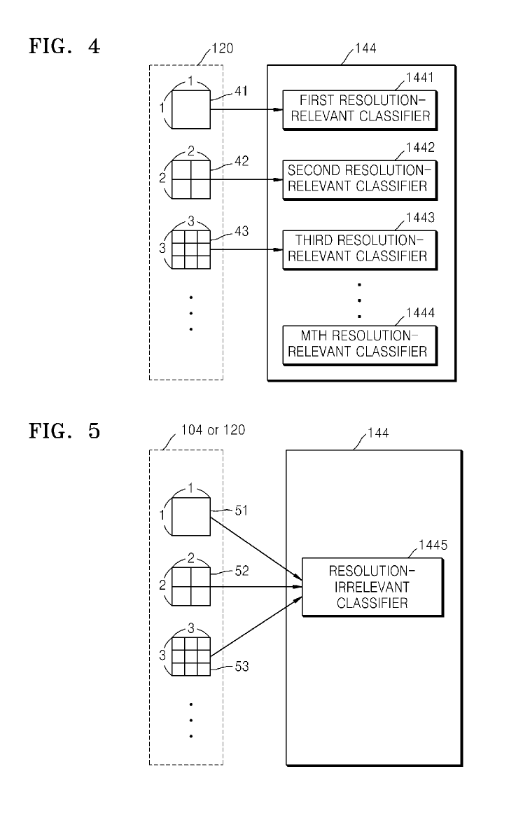

However, when the second determination unit 144 determines the second feature value for determining whether the first tissue has a lesion or has no lesion, the second feature value may be determined according to the resolution of the ROI included in the emphatic image, or a feature commonly extracted from the diagnostic image and the emphatic image regardless of the resolution. A method of determining the second feature value will be described in detail later with reference to FIGS. 4 and 5.

Further, a classifier included in the second determination unit 144 may be a classifier using a CAD method. As described above in relation to the first determination unit 142, the classifier using learned data in the CAD method may be known to one of ordinary skill in the art that, and thus a detailed description thereof is not provided here.

In addition, the second determination unit 144 compares the second feature value to a threshold value for determining a lesion and determines that the first tissue has a lesion if the second feature value is greater than the threshold value. For example, the classifier included in the second determination unit 144 may classify the first tissue as having a lesion.

On the other hand, the second determination unit 144 determines that the first tissue has no lesion if the second feature value is equal to or less than the threshold value. For example, the classifier included in the second determination unit 144 may classify the first tissue as having no lesion.

In addition, the threshold value used by the second determination unit 144 may generally be, but is not limited to, the same as the threshold value used by the first determination unit 142.

If a determination result of the first determination unit 142 is different from the determination result of the second determination unit 144, the third determination unit 146 determines whether the first tissue has a lesion by mixing the first and second feature values according to a determination ratio.

However, if the first and second determination units 142 and 144 have the same determination result, the third determination unit 146 determines the same determination result as a final result.

For example, if the first determination unit 142 determines the first tissue included in the first ROI has a lesion and the second determination unit 144 determines the first tissue included in the first ROI has a lesion, the third determination unit 146 determines the first tissue included in the first ROI has a lesion.

However, if the first determination unit 142 determines the first tissue included in the first ROI as having a lesion and the second determination unit 144 does not determine the first tissue included in the first ROI as having a lesion, or if the first determination unit 142 does not determine the first tissue included in the first ROI as having a lesion and the second determination unit 144 determines the first tissue included in the first ROI as having a lesion, the third determination unit 146 determines whether the first tissue has a lesion by mixing the first and second feature values according to a determination ratio. Further, the determination ratio refers to a ratio for mixing the first and second feature values and may be set in default or by a user.

As such, the third determination unit 146 may perform calculation as shown in Equation 1.

FV_{Final}=R₁×FV₁+(1−R₁)FV₂ <Equation 1>

In Equation 1, FV_{Final} is a final feature value, FV₁ is a first feature value, FV₂ is a second feature value, and R₁ is a value for setting a determination ratio and may be a rational number equal to or greater than 0 and equal to or less than 1. As such, the determination ratio may be R₁:(1−R₁).

Accordingly, a user may adjust the determination ratio by setting R₁. The user may increase R₁ if the reliability on a result in the diagnostic image is higher than that in the emphatic image, and may reduce R₁ if the reliability on a result in the emphatic image is higher than that in the diagnostic image.

As such, the third determination unit 146 calculates the final feature value as shown in Equation 1, compares the calculated final feature value to a threshold value for determining a lesion, and determines whether the first tissue has a lesion.

That is, the third determination unit 146 compares the final feature value to the threshold value for determining a lesion, and determines that the first tissue has a lesion if the final feature value is greater than the threshold value.

On the other hand, the third determination unit 146 determines that the first tissue has no lesion if the final feature value is equal to or less than the threshold value.

The threshold value used by the third determination unit 146 may generally be, but is not limited to, the same as the threshold value used by the first and second determination units 142 and 144.

For example, if the first feature value regarding the first tissue included in the first ROI is 3.4, the second feature value is 3.6, and the threshold value for determining a lesion is 3.5, the first determination unit 142 determines that the first tissue included in the first ROI has no lesion, and the second determination unit 144 determines that the first tissue included in the first ROI has a lesion.

Further, the third determination unit 146 determines whether the first tissue has a lesion by mixing the first and second feature values according to a determination ratio. If the value R₁ for setting the determination ratio is 0.4, the third determination unit 146 may perform calculation as shown in Equation 2.

FV_{Final}=0.4×3.4+(1−0.4)×3.6=3.52 <Equation 2>

As such, since the final feature value is greater than the threshold value, i.e., 3.5, the third determination unit 146 determines that the first tissue included in the first ROI has a lesion.

Accordingly, the diagnostic apparatus 100 may accurately and automatically determine whether a tissue included in an ROI has a lesion, and, thus, may serve to improve convenience and accuracy of diagnosis.

In addition, the second determination unit 144 may determine whether the first tissue included in the first ROI has a lesion by using two or more emphatic images. For example, if the emphatic image generation unit 120 generates a plurality of emphatic images in which the resolutions of a normal region and one or more ROIs are different, the second determination unit 144 may determine whether the first tissue included in the first ROI has a lesion in each of the emphatic images.

As such, the third determination unit 146 may determine whether the first tissue included in the first ROI has a lesion in consideration of a determination result of the first determination unit 142 and a plurality of determination results of the second determination unit 144.

For example, if N emphatic images are generated, the third determination unit 146 may perform calculation as shown in Equation 3.

$${FV}_{Final} = {R}_{1} \times {FV}_{1} + {\sum }_{n = 1}^{N} \frac{1 - {R}_{1}}{N} {FV}_{2 n} \qquad < Equation 3 >$$

In Equation 3, FV_{Final} is a final feature value, FV₁ is a first feature value, FV₂ₙ is a second feature value regarding an nth emphatic image, and R₁ is a value for setting a determination ratio and may be a rational number equal to or greater than 0 and equal to or less than 1. Further, n and N are natural numbers, and N may be a natural number equal to or greater than 1.

As such, the diagnostic apparatus 100 may determine whether a tissue included in an ROI has a lesion by using a plurality of emphatic images, and, thus, may server to improve accuracy of a diagnosis result.

Accordingly, the lesion determination unit 140 may determine whether a tissue included in each ROI has a lesion or has no lesion. Further, the display unit 130 may display a determination result of the lesion determination unit 140 together with an emphatic image.

For example, from among first through third ROIs included in the emphatic image, if only the first ROI has a lesion, the display unit 130 displays that the first ROI has a lesion.

As such, a user may intuitively recognize whether a subject has a lesion. Thus, the user's utilization of the display unit 130 may serve to improve convenience of diagnosis.

The database 150 stores information regarding features for detecting an ROI. The feature for detecting an ROI may include a size, a shape, a margin, and a calcification level of a tissue.

If the lesion determination unit 140 determines that the first tissue has no lesion, the database management unit 155 adds to the database 150 information representing that a feature of the first tissue does not correspond to an ROI.

An example is now described when a tissue included in the first ROI has a size of about 0.2×0.2 cm², an oval shape, and a stellate margin. Although the ROI detection unit 110 detects the first ROI, if the lesion determination unit 140 determines that the first ROI has no lesion, the database management unit 155 adds to the database 150 information representing that a feature of the first tissue included in the first ROI does not correspond to an ROI.

That is, if the first tissue has a feature such as a size of about 0.2×0.2 cm², an oval shape, and a stellate margin, the database management unit 155 adds to the database 150 information representing that the first tissue is not an ROI.

As such, the database management unit 155 may improve the accuracy of detecting an ROI by the ROI detection unit 110 of the diagnostic apparatus 100.

In addition, since the diagnostic apparatus 100 automatically generates an emphatic image in which the resolution of an ROI required to be attentively observed in a diagnostic process is improved, diagnosis may be performed rapidly and accurately and accuracy of diagnosis may be ensured regardless of the experience and prior knowledge of a user of the diagnostic apparatus 100.

FIG. 4 is a block diagram illustrating an example of the second determination unit 144 illustrated in FIG. 3. Referring to FIG. 4, the second determination unit 144 includes a plurality of resolution-relevant classifiers for classifying a tissue included the ROI as having a lesion or having no lesion in correspondence with each of a plurality of available resolutions of an ROI included in an emphatic image.

The resolution-relevant classifiers may include a first classifier 1441, a second classifier 1442, a third classifier 1443, . . . , and an Mth classifier 1444.

For example, as illustrated in FIG. 4, if an ROI 41 has a resolution of 1×1, the first classifier 1441 determines a second feature value indicating a level of a feature representing whether a first tissue included in the ROI 41 has a lesion, and classifies the first tissue as having a lesion or having no lesion by using the determined second feature value.

As another example, if an ROI 42 has a resolution of 2×2, the second classifier 1442 determines a second feature value indicating a level of a feature representing whether a first tissue included in the ROI 42 has a lesion, and classifies the first tissue as having a lesion or having no lesion by using the determined second feature value.

As still another example, if an ROI 43 has a resolution of 3×3, the third classifier 1443 determines a second feature value indicating a level of a feature representing whether a first tissue included in the ROI 43 has a lesion, and classifies the first tissue as having a lesion or having no lesion by using the determined second feature value.

In this manner, the second determination unit 144 may use one of a plurality of resolution-relevant classifiers to determine whether a tissue included in the ROI has a lesion in correspondence with each of the resolutions of each ROI included in an emphatic image, and may classify the tissue as having a lesion or having no lesion according to a determination result.

FIG. 5 is a block diagram illustrating another example of the second determination unit 144 illustrated in FIG. 3. Referring to FIG. 5, the second determination unit 144 includes a resolution-irrelevant classifier 1445 that extracts features representing whether a first tissue has a lesion commonly from a diagnostic image and an emphatic image, and classifies the first tissue as having a lesion or having no lesion by using the extracted features.

For example, as illustrated in FIG. 5, the resolution-irrelevant classifier 1445 extracts features representing whether a first tissue included in a first ROI has a lesion commonly from the first ROI included in the diagnostic image and the first ROI included in the emphatic image. Further, the resolution-irrelevant classifier 1445 extracts the features regarding the first tissue from the diagnostic image and the emphatic image, and classifies the first tissue as having a lesion or having no lesion by using the extracted feature.

For example, if the first ROI 51 included in the diagnostic image has a resolution of 1×1, and the first ROI 52 included in the emphatic image has a resolution of 2×2, the resolution-irrelevant classifier 1445 extracts features representing whether a first tissue included in the first ROI 51 and 52 has a lesion commonly from the first ROI 51 having a resolution of 1×1 and included in the diagnostic image, and the first ROI 52 having a resolution of 2×2 and included in the emphatic image. In addition, the resolution-irrelevant classifier 1445 determines a second feature value by using the extracted features, and classifies the first tissue as having a lesion or having no lesion by using the determined second feature value.

In this manner, the second determination unit 144 may determine whether a tissue included in an ROI has a lesion by using the resolution-irrelevant classifier 1445, and may classify whether the tissue has a lesion or has no lesion according to a determination result.

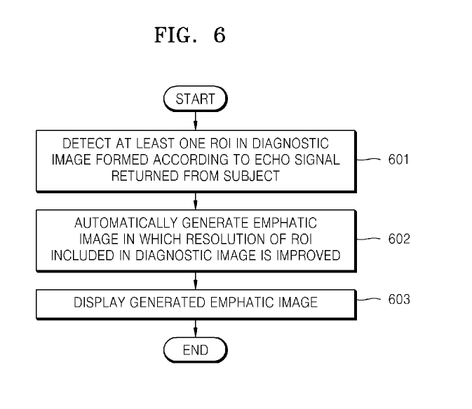

FIG. 6 is a flowchart illustrating an example of a diagnostic method according to a general aspect. Referring to FIG. 6, the diagnostic method includes operations performed in time series by the diagnostic apparatus 100 illustrated in FIGS. 1 and 3. Accordingly, descriptions made above in relation to the diagnostic apparatus 100 may also be applied to the diagnostic method and may not be provided here.

In operation 601, the ROI detection unit 110 detects one or more ROIs in a diagnostic image formed according to an echo signal returned from a subject.

In operation 602, if the ROI is detected in operation 601, the emphatic image generation unit 120 automatically generates an emphatic image in which the resolution of the ROI included in the diagnostic image is improved. In addition, the emphatic image generation unit 120 may generate an emphatic image in which the resolutions of a plurality of ROIs are improved to different ratios according to a level of a feature representing whether a tissue included in each of the ROIs has a lesion. In addition, the emphatic image generation unit 120 may generate the emphatic image in which the resolution of the ROI is increased to a higher ratio if there is a high probability that a tissue included in the ROI has a lesion.

In operation 603, the display unit 130 displays the emphatic image generated in operation 602.

According to the diagnostic method, an emphatic image in which the resolution of an ROI of a subject is improved may be automatically generated.

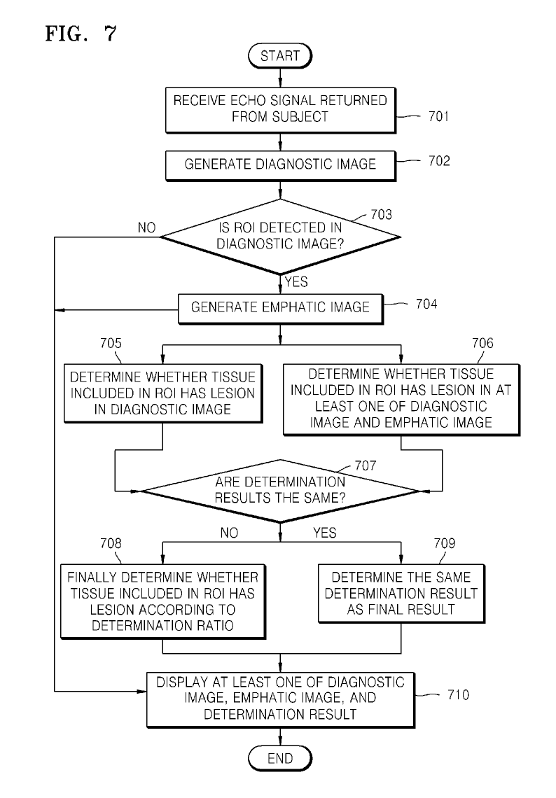

FIG. 7 is a flowchart illustrating an example of a diagnostic method according to another general aspect. Referring to FIG. 7, the diagnostic method includes operations performed in time series by the diagnostic apparatus 100 illustrated in FIGS. 1 and 3. Accordingly, descriptions made above in relation to the diagnostic apparatus 100 may also be applied to the diagnostic method and may not be provided here.

In operation 701, the probe 102 receives an echo signal returned from a subject.

In operation 702, the diagnostic image generation unit 104 generates a diagnostic image by using the echo signal received in operation 701.

In operation 703, the ROI detection unit 110 attempts to detect one or more ROIs in the diagnostic image generated in operation 702. Further, the diagnostic method proceeds to operation 710 if the ROI is not detected, and proceeds to operation 704 if the ROI is detected.

In operation 704, if the ROI is detected in operation 703, the emphatic image generation unit 120 automatically generates an emphatic image in which the resolution of the ROI included in the diagnostic image is improved.

In operation 705, the first determination unit 142 determines whether a tissue included in each ROI has a lesion in the diagnostic image. In operation 706, the second determination unit 144 determines whether the tissue included in each ROI has a lesion in the diagnostic image, the emphatic image, or a combination thereof.

In operation 707, the third determination unit 146 determines whether a determination result of operation 705 is the same as a determination result of operation 706. The diagnostic method proceeds to operation 708 if the determination results of operations 705 and 706 are not the same, and proceeds to operation 709 if the determination results of operations 705 and 706 are the same.

In operation 708, the third determination unit 146 determines whether the tissue included in the ROI has a lesion by mixing, according to a determination ratio, a first feature value used by the first determination unit 142 in operation 705 and a second feature value used by the second determination unit 144 in operation 706.

In operation 709, the third determination unit 146 determines the same determination result as a final result.

In operation 710, the display unit 130 displays the diagnostic image, the emphatic image, the determination result, or any combination thereof. Further, the display unit 130 may display the diagnostic image, the emphatic image, the determination result, or any combination thereof on one screen.

As such, according to the diagnostic method, an automatic determination and display of whether a tissue included in an ROI of a subject has a lesion may be made. Thus, a user may intuitively recognize a diagnosis result. In addition, since whether the tissue included in the ROI has a lesion is determined in consideration of both a diagnostic image and an emphatic image, accuracy of diagnosis may be improved.

According to teachings above, there is provided the automatic generation of a high-resolution image that may allow a user to easily determine whether a subject has a lesion.

According to teachings above, there is provided diagnostic apparatuses and methods that may be capable of automatically generating a high-resolution image of a region of interest (ROI).

Program instructions to perform a method described herein, or one or more operations thereof, may be recorded, stored, or fixed in one or more computer-readable storage media. The program instructions may be implemented by a computer. For example, the computer may cause a processor to execute the program instructions. The media may include, alone or in combination with the program instructions, data files, data structures, and the like. Examples of computer-readable media include magnetic media, such as hard disks, floppy disks, and magnetic tape; optical media such as CD ROM disks and DVDs; magneto-optical media, such as optical disks; and hardware devices that are specially configured to store and perform program instructions, such as read-only memory (ROM), random access memory (RAM), flash memory, and the like. Examples of program instructions include machine code, such as produced by a compiler, and files containing higher level code that may be executed by the computer using an interpreter. The program instructions, that is, software, may be distributed over network coupled computer systems so that the software is stored and executed in a distributed fashion. For example, the software and data may be stored by one or more computer readable recording mediums. Also, functional programs, codes, and code segments for accomplishing the example embodiments disclosed herein can be easily construed by programmers skilled in the art to which the embodiments pertain based on and using the flow diagrams and block diagrams of the figures and their corresponding descriptions as provided herein. Also, the described diagnostic apparatus to perform an operation or a method may be hardware, software, or some combination of hardware and software. For example, the diagnostic apparatus may be a software package running on a computer or the computer on which that software is running.

A number of examples have been described above. Nevertheless, it will be understood that various modifications may be made. For example, suitable results may be achieved if the described techniques are performed in a different order and/or components in a described system, architecture, device, or circuit are combined in a different manner and/or replaced or supplemented by other components or their equivalents. Accordingly, other implementations are within the scope of the following claims.

What is claimed is:

1. A diagnostic apparatus, comprising:
a region of interest (ROI) detection unit configured to detect at least one ROI in a diagnostic image formed according to an echo signal returned from a subject;
an emphatic image generation unit configured to automatically generate an emphatic image in which a resolution of the detected ROI is improved; and
a display unit configured to display the generated emphatic image.

2. The diagnostic apparatus of claim 1, wherein:
the ROI detection unit configured to detect a plurality of ROIs, and
the emphatic image generation unit is further configured to generate the emphatic image in which the resolution of each of the detected ROIs is improved according to different ratios corresponding to a level of a feature of representing whether a tissue included in each of the ROIs has a lesion.

3. The diagnostic apparatus of claim 1, wherein the emphatic image generation unit is further configured to generate the emphatic image in which the resolution of the detected ROI is increased to a higher ratio if a probability is high that a tissue included in the detected ROIs has a lesion.

4. The diagnostic apparatus of claim 1, further comprising:
a lesion determination unit configured to:
determine whether a first tissue included in a first ROI has a lesion in the diagnostic image, the emphatic image, or a combination thereof with respect to each of the detected ROI; and
determine whether the first tissue has a lesion by using a determined result.

5. The diagnostic apparatus of claim 4, wherein the lesion determination unit comprises:
a first determination unit configured to determine whether the first tissue has a lesion by using a first feature value indicating a level of a feature representing whether the first tissue has a lesion in the diagnostic image;
a second determination unit configured to determine whether the first tissue has a lesion by using a second feature value indicating a level of a feature representing whether the first tissue has a lesion in at least one of the diagnostic image and the emphatic image; and
a third determination unit configured to determine whether the first tissue has a lesion by mixing the first and second feature values according to a determination ratio if a result of the first determination unit is different from a result of the second determination unit.

6. The diagnostic apparatus of claim 5, wherein the second determination unit is further configured to determine whether the first tissue has a lesion by using two or more emphatic images.

7. The diagnostic apparatus of claim 5, wherein the second determination unit comprises a plurality of resolution-relevant classifiers configured to classify the first tissue as having a lesion or having no lesion in correspondence with each of a plurality of available resolutions of the ROI included in the emphatic image.

8. The diagnostic apparatus of claim 5, wherein the second determination unit comprises:
a resolution-irrelevant classifier configured to:
extract features representing whether the first tissue has a lesion commonly from the diagnostic image and the emphatic image; and
classify the first tissue as having a lesion or having no lesion by using the extracted features.

9. The diagnostic apparatus of claim 4, further comprising:
a database configured to store information regarding features for detecting the ROI; and
a database management unit configured to add to the database information representing that a feature of the first tissue does not correspond to the ROI if the lesion determination unit determines that the first tissue has no lesion.

10. The diagnostic apparatus of claim 4, wherein the lesion determination unit automatically determines whether the tissue included in the detected ROI has a lesion.

11. The diagnostic apparatus of claim 4, wherein the display unit is configured to display a result of the determination of the lesion determination unit together with the emphatic image.

12. A diagnostic method, comprising:
detecting at least one region of interest (ROI) in a diagnostic image formed according to an echo signal returned from a subject;
automatically generating an emphatic image in which a resolution of the detected ROI is improved; and
displaying the generated emphatic image.

13. The diagnostic method of claim 12, wherein:
the detecting of the ROI comprises detecting a plurality of ROIs, and
the automatic generating of the emphatic image comprises automatically generating the emphatic image in which the resolutions of each of the detected ROIs is improved according to different ratios corresponding to a level of a feature representing whether a tissue included in each of the ROIs has a lesion.

14. The diagnostic method of claim 12, wherein the automatic generating of the emphatic image comprises automatically generating the emphatic image in which the resolution of the detected ROI is increased to a higher ratio if a probability is high that a tissue included in the detected ROI has a lesion.

15. The diagnostic method of claim 12, further comprising:
determining whether a first tissue included in a first ROI has a lesion in the diagnostic image, the emphatic image, or a combination thereof with respect to each of the detected ROI; and
determining whether the first tissue has a lesion by using a determined result.

16. The diagnostic method of claim 12, further comprising:
determining whether the first tissue has a lesion by using a first feature value indicating a level of a feature representing whether the first tissue has a lesion in the diagnostic image;
determining whether the first tissue has a lesion by using a second feature value indicating a level of a feature representing whether the first tissue has a lesion in the diagnostic image, and the emphatic image, or a combination thereof; and
determining whether the first tissue has a lesion by mixing the first and second feature values according to a determination ratio, if a result obtained by using the first feature value is different from a result obtained by using the second feature value.

17. The diagnostic method of claim 16, wherein the determining of whether the first tissue has a lesion by using the second feature value comprises determining whether the first tissue has a lesion by using two or more emphatic images.

18. The diagnostic method of claim 16, wherein the determining of whether the first tissue is a lesion by using the second feature value comprises determining whether the first tissue is a lesion by using a plurality of resolution-relevant classifiers configured to classify the first tissue as having a lesion or having no lesion in correspondence with each of a plurality of available resolutions of the ROI included in the emphatic image.

19. The diagnostic method of claim 16, wherein the determining of whether the first tissue has a lesion by using the second feature value comprises determining whether the first tissue has a lesion by using a resolution-irrelevant classifier configured to extract features representing whether the first tissue has a lesion commonly from the diagnostic image and the emphatic image, and classify the first tissue as having a lesion or having no lesion by using the extracted features.

20. A computer readable recording medium having recorded thereon a computer program for executing the diagnostic method of claim 12.
